(19) United States
(12) Patent Application Publication
(10) Pub. No.: US 2026/0232665 A1
(43) Pub. Date: Aug. 13, 2026

(54) PHARMACEUTICAL COMPOSITION CONTAINING MINOXIDIL, PREPARATION METHOD AND USE THEREOF

(71) Applicant: Auson Pharmaceuticals, Inc, Bridgewater, NJ (US)

(72) Inventors: Peng HOU, Shanghai (CN); Enxian LU, Shanghai (CN); Pingwei WANG, Shanghai (CN)

(21) Appl. No.: 19/636,688

(22) Filed: Apr. 1, 2026

Related U.S. Application Data
(63) Continuation-in-part of application No. PCT/CN2025/084603, filed on Mar. 25, 2025.

(30) Foreign Application Priority Data
Mar. 28, 2024 (CN) 202410362086.6

(51) Int. Cl. A61K 31/506; A61K 9/20

(52) U.S. Cl. CPC A61K 31/506; A61K 9/2009; A61K 9/2013; A61K 9/2018; A61K 9/2031; A61K 9/2054

(57) ABSTRACT

Provided are a solid pharmaceutical composition and a preparation method thereof. The solid pharmaceutical composition includes: a) a therapeutically effective amount of minoxidil; b) a pharmaceutically acceptable release controlling agent; c) a lubricant, d) a filler, and e) a glidant, where the therapeutically effective amount is in a range of 1 mg to 10 mg; and the solid pharmaceutical composition exhibits the following in vitro release profile when tested using a paddle method at a rotational speed of 75 rpm and in 900 mL of a phosphate buffer solution with a pH of 7.2: at 1 hour, 15% to 30% of the minoxidil is released; at 4 hours, 55% to 75% of the minoxidil is released; and at 8 hours, more than 80% of the minoxidil is released.

### CROSS REFERENCE TO RELATED APPLICATION

The present application is a continuation-in-part of International Patent Application No. PCT/CN2025/084603, filed on Mar. 25, 2025, which claims priority to Chinese Patent Application No. CN202410362086.6, filed with the China National Intellectual Property Administration (CNIPA) on Mar. 28, 2024 and entitled “PHARMACEUTICAL COMPOSITION CONTAINING MINOXIDIL, PREPARATION METHOD AND USE THEREOF”. The disclosure of the two applications is incorporated herein by reference in its entirety.

### TECHNICAL FIELD

The present disclosure belongs to the pharmaceutical field, and in particular relates to a pharmaceutical composition containing minoxidil, a preparation method and use thereof.

### BACKGROUND

Androgenic alopecia (AGA), previously known as seborrheic alopecia or premature alopecia, is a progressive hair loss disease that occurs during adolescence and post-adolescence. In males, it primarily manifests as frontal hairline recession and/or progressive thinning and reduction of hair on the vertex, also known as male pattern alopecia. In females, it mainly manifests as progressive thinning and reduction of hair on the vertex, with a small proportion showing diffuse hair thinning without hairline recession, referred to as female pattern alopecia. The prevalence of this disease varies significantly among different ethnic groups, with a higher incidence in Caucasians and lower incidences in Black and Asian populations. The latest epidemiological survey shows that the prevalence of this disease is 21.3% in males and 6.0% in females. This disease has a significant impact on patients' mental health and quality of life. If diagnosed early and treated correctly, most patients can experience improvement.

Minoxidil is a potassium channel opener. Potassium channel opening is an important step in regulating hair growth. Both in vivo and in vitro experiments in animal models show that minoxidil is a potassium channel activator, which can increase the permeability of potassium ions and prevent calcium ion influx into cells, resulting in a decrease in the concentration of free calcium ions within the cells. In the presence of calcium ions, epidermal growth factor inhibits hair growth.

Minoxidil was first developed by Pfizer and approved for marketing in the United States in October 1979. The marketed dosage form was tablets, with strengths of 2.5 mg and 10 mg, respectively, which are used to treat adult refractory hypertension, with hirsutism being its main side effect. Based on this discovery, Johnson Company developed a topical minoxidil solution for the treatment of hair loss in both males and females. The marketed strength was 2%, and it was approved for marketing in the United States in August 1988. In November 1997, Johnson developed a 5% topical solution for the treatment of male hair loss. In 2006, Johnson improved the solution preparation and developed a minoxidil foam with a strength of 5%. Currently, the dosage forms of minoxidil for the treatment of hair loss that have been approved for marketing both domestically and internationally are all external dosage forms. It can be seen that although some studies have found that oral LDOM at 1.25 mg/d can effectively improve hair loss, there are currently no oral preparations of minoxidil specifically indicated for hair loss on the market.

The inventors discovered through research that the innovator conducted a trial on the tablet to investigate the serum concentration of minoxidil and systemic safety. The results proved that no systemic safety issues arise when the serum concentration of minoxidil is below 20 ng/mL. Furthermore, the development target for the topical minoxidil solution preparation was also established based on this limit. When the conventional oral minoxidil tablet of 2.5 mg/tablet is used for treating refractory hypertension, the serum concentration of minoxidil exceeds 20 ng/mL. Oral tablets with a strength lower than 2.5 mg are considered low-dose tablets, which carry a higher risk of content uniformity during the manufacturing process.

Tablets have many advantages over topical scalp administration, such as oral preparations typically contain no solvents, are more convenient to use, can accurately control the dose, and do not need to consider scalp absorption. However, the development of minoxidil tablets still faces significant technical challenges, particularly how to ensure adequate systemic exposure while reducing the minoxidil concentration. To achieve this object, a minoxidil sustained-release preparation is preferred.

Developing an oral sustained-release preparation containing minoxidil that simultaneously possesses safety, efficacy, and preparation stability, thereby improving the compliance of patients, has become a product and technological gap in this field.

### SUMMARY

In view of the above, the inventors provide a pharmaceutical composition containing minoxidil, preferably a sustained-release pharmaceutical composition. Compared with immediate-release tablets, sustained-release tablets can reduce the serum concentration (Cmax) of minoxidil, which is related to systemic safety. The U.S. Food and Drug Administration (FDA) considers that the Cmax lower than 20 ng/ml will not exhibit the pharmacological effect of hypertension. To further enhance safety, the inventors set a development target for the minoxidil sustained-release tablet to be no higher than 10 ng/mL, while maintaining a bioavailability similar to that of conventional tablets. This brings more challenges for the development of oral medicaments.

Meanwhile, it is reported that over 90% of minoxidil is absorbed through the gastrointestinal tract. Therefore, it can be inferred that minoxidil is well absorbed in the upper part of the small intestine. Consequently, developing a particular sustained-release dosage form may require further exploration. Since there are no minoxidil sustained-release tablet dosage forms on the market, the absorption of minoxidil throughout the gastrointestinal tract is unclear; in particular, whether the drug is absorbed in the colon is also unknown. Thus, how to adjust the release mode of sustained-release tablets and related issues of bioavailability need to be solved urgently.

To achieve the above technical objects, namely, to develop an ideal pharmaceutical composition containing minoxidil that possesses safety and good bioavailability, the present disclosure provides a solid pharmaceutical composition, including:
a) a therapeutically effective amount of minoxidil,
b) a pharmaceutically acceptable release controlling agent,
c) a lubricant,
d) a filler, and
e) a glidant, where
the therapeutically effective amount is in a range of 1 mg to 10 mg; and
the solid pharmaceutical composition exhibits the following in vitro release profile when tested using a paddle method at a rotational speed of 75 rpm and in 900 mL of a phosphate buffer solution with a pH of 7.2:
at 1 hour, 15% to 30% of the minoxidil is released;
at 4 hours, 55% to 75% of the minoxidil is released; and
at 8 hours, more than 80% of the minoxidil is released.

In some embodiments, in the solid pharmaceutical composition, the therapeutically effective amount is in a range of 2 mg to 8 mg.

In some embodiments, in the solid pharmaceutical composition, the therapeutically effective amount is 3.0 mg, 3.1 mg, 3.2 mg, 3.3 mg, 3.4 mg, 3.5 mg, 3.6 mg, 3.7 mg, 3.8 mg, 3.9 mg, 4.0 mg, 4.1 mg, 4.2 mg, 4.3 mg, 4.4 mg, 4.5 mg, 4.6 mg, 4.7 mg, 4.8 mg, 4.9 mg, 5.0 mg, 5.1 mg, 5.2 mg, 5.3 mg, 5.4 mg, 5.5 mg, 5.6 mg, 5.7 mg, 5.8 mg, 5.9 mg, or 6.0 mg twice daily.

In some embodiments, in the solid pharmaceutical composition, the solid pharmaceutical composition is in a form of a tablet, a capsule, or a suspension.

In some embodiments, in the solid pharmaceutical composition, the solid pharmaceutical composition is in a form of the tablet.

In some embodiments, in the solid pharmaceutical composition, the solid pharmaceutical composition consists of: in percentage by weight, 0.1%-5% of the minoxidil, 30%-50% of the pharmaceutically acceptable release controlling agent, 0.1%-2% of the glidant, 0.1%-2% of the lubricant, and the remaining amount of the filler.

In some embodiments, in the above solid pharmaceutical composition, the solid pharmaceutical composition consists of: in percentage by weight, 0.5%-2.5% of the minoxidil, 35%-45% of the pharmaceutically acceptable release controlling agent, 0.1%-1% of the glidant, 0.1%-1% of the lubricant, and the remaining amount of the filler.

In some embodiments, in the solid pharmaceutical composition, the filler contains a hydrophobic component and a hydrophilic component.

In some embodiments, in the solid pharmaceutical composition, the hydrophobic component is one or more selected from the group consisting of microcrystalline cellulose and calcium hydrogen phosphate.

In some embodiments, in the solid pharmaceutical composition, the hydrophilic component is one or more selected from the group consisting of lactose and xylitol.

In some embodiments, in the solid pharmaceutical composition, the pharmaceutically acceptable release controlling agent is one or more selected from the group consisting of hypromellose, hydroxypropyl cellulose, ethyl cellulose, polyethylene oxide, carbomers and methacrylic acid copolymer.

In some embodiments, in the solid pharmaceutical composition, the pharmaceutically acceptable release controlling agent is the hypromellose.

In some embodiments, in the solid pharmaceutical composition, the glidant is one or more selected from the group consisting of colloidal silica and talc.

In some embodiments, in the solid pharmaceutical composition, the lubricant is one or more selected from the group consisting of magnesium stearate and sodium stearyl fumarate.

In some embodiments, in the solid pharmaceutical composition, the filler is a combination of microcrystalline cellulose and lactose in a weight ratio of 1-5:1.

In some embodiments, in the solid pharmaceutical composition, the filler is a combination of microcrystalline cellulose and lactose in a weight ratio of 2-4.81:1.

In some embodiments, in the above method, the solid pharmaceutical composition is administered to a patient that has been diagnosed with hair loss.

The present disclosure further provides a method for preparing the solid pharmaceutical composition as mentioned above, comprising (consisting of):
1) mixing the therapeutically effective amount of the minoxidil with a part of the filler in such amount that a volume ratio of the minoxidil to a hydrophobic component of the filler ranges from 1:2 to 1:5 to obtain a material 1;
2) mixing a remainder of the filler with the material 1 in step 1), and then subjecting a resulting mixture to sieving with a granulate machine to obtain a material 2;
3) subjecting the glidant and the pharmaceutically acceptable release controlling agent to sieving with the granulate machine, and then mixing a resulting material with the material 2 in step 2) to obtain a material 3; and
4) adding the lubricant to the material 3 obtained from step 3) and then mixing to obtain the solid pharmaceutical composition.

### Technical Objects:

1. The average maximum plasma concentration is lower than 10 ng/mL, which is lower than half of the safe concentration limit of 20 ng/mL.

Compared with the same dose of an immediate-release tablet, the relative bioavailability is maximized, which allows for dose reduction and safety enhancement.
2. Compared with a minoxidil solution, a similar systemic exposure (AUC) is provided, which improves the safety and effectiveness of minoxidil sustained-release tablets.

### Technical Advantages:

1. Sustained-release tablets can significantly reduce the maximum plasma concentration in vivo (lower than 10 ng/mL), thereby improving the safety of oral administration.
2. Sustained-release tablets can provide patients with an alternative choice for scalp administration.
3. The in vitro 8-hour sustained-release effect shows both a reduction in the maximum plasma concentration and sustained drug release, resulting in sustained efficacy.
4. The tablet prepared according to the present disclosure has stable quality, small difference in in vitro release, and good safety.
5. The tablet prepared according to the present disclosure has good stability and can meet a shelf life of at least 2 years over the long term.

The minoxidil sustained-release tablet is tested using the USP apparatus 2 paddle method, with a rotational speed of 75 rpm, a phosphate buffer with a medium pH of 7.2 in a volume of 900 mL, and can achieve an 8-hour sustained-release effect, with a release of 15% to 35% at 1 hour, 50% to 70% at 4 hours, and not less than 80% at 8 hours, or with a release of 15% to 30% at 1 hour, 55% to 75% at 4 hours, and not less than 80% at 8 hours.

It can be found from the in vivo experiments, the separate study of the release controlling agent and the sustained-release time reveal their significant effect on bioavailability, which is rarely addressed in other studies. For example, the inventors found that different release controlling agents, even with identical in vitro release, resulted in inconsistent systemic exposure in vivo; the bioavailability of the 8-hour sustained-release tablet relative to the immediate-release tablet is higher than that of the 12-hour sustained-release tablet. Concurrently, the inventors discovered that the formulation containing mannitol exhibited reduced release during the accelerated and long-term stability studies. The inventors performed a compatibility study between minoxidil and different fillers, ultimately concluding that mannitol and minoxidil are incompatible, and lactose is identified as the alternative excipient due to its similarity to mannitol in solubility and hydrophilicity.

The strength issue of low-dose drugs in the present disclosure has been solved. Note: Since the proportion of the minoxidil in the formulation is less than 2.5%, it is classified as a “high-potency” drug. The content uniformity of the tablet is a key internal control parameter. The inventors investigated the effect of different processes on the content uniformity of the tablet, and ultimately established the method for preparing the minoxidil sustained-release tablet, which belong to the content not previously covered in minoxidil research and are encountered for the first time.

### BRIEF DESCRIPTION OF THE DRAWINGS

FIG. 1 shows dissolution curves of different release controlling agents;

FIG. 2 shows dissolution curves of different sustained-release behaviors;

FIG. 3 shows dissolution curves of Formulation 6 under different conditions;

FIG. 4 shows dissolution curves at different HPMC levels;

FIG. 5 shows the composition of formulations with different microcrystalline cellulose/lactose ratios; and

FIG. 6 shows dissolution curves at different doses.

### DETAILED DESCRIPTION OF THE EMBODIMENTS

In the present disclosure, the lubricant, the filler, and the glidant are collectively referred to as excipients; the excipients could further include other additives. In the present disclosure, there are no specific limitations on the other additives, and any additives that could be used as excipients known to those skilled in the art may be adopted.

In the present disclosure, the filler is also known as a diluent.

The following examples are provided to further illustrate the present disclosure, but are not intended to limit the present disclosure.

In the following examples, lactose was supplied by MEGGLE; hypromellose was supplied by Colorcon; mannitol was supplied by Roquette; microcrystalline cellulose was supplied by FMC; calcium hydrogen phosphate was supplied by Spectrum; hydroxypropyl cellulose was supplied by Ashland; croscarmellose sodium was supplied by JRS; colloidal silica was supplied by EVONIK, polyethylene oxide (PEO) was supplied by Danisco; and magnesium stearate was supplied by FACI.

### Example 1—Investigation of Different Release Controlling Agents

Different release controlling agents can all achieve identical in vitro dissolution behaviors. However, due to their different hydrophilicity and the influence of gastrointestinal motility, different release controlling agents may exhibit different in vivo performance. The inventors selected three release controlling agents for the development of minoxidil sustained-release tablets and also developed a 2.5 mg immediate-release tablet to compare the relative bioavailability of different sustained-release matrices with that of the immediate-release tablet. The composition of formulation for the sustained-release tablet is shown in Table 1, and the composition of formulation for the immediate-release tablet is shown in Table 2.

The formulation structure adopted by the inventors consisted of a filler, a release controlling agent, a glidant, and a lubricant, where the glidant and the lubricant were colloidal silica and magnesium stearate, respectively. Microcrystalline cellulose is insoluble in water, is a plastic material, and has good compressibility. Mannitol is soluble in water, is a brittle material, and has good flowability. During the formulation development, the two are often combined as a filler to confer good compressibility and flowability to the drug powder. Therefore, this product selected the combination of the two as the filler of the present disclosure.

TABLE 1
Composition of formulations with different release controlling agents

| | Formulation 1- | Formulation 2- | Formulation 3- | |
|---|---|---|---|---|
| | HPMC K4M | HPC | PEO | |
| Component | mg/tablet | | | Function |
| Minoxidil | 2.5 | 2.5 | 2.5 | Active ingredient |
| Mannitol 200SD | 20 | 24.5 | 14 | Filler |
| Microcrystalline | 95.5 | 100 | 89.5 | Filler |
| cellulose PH102 | | | | |
| Hypromellose K4M | 80 | / | / | Release |
| | | | | controlling agent |
| Polyethylene Oxide | / | / | 92 | Release |
| (PEO WSR-N-60K) | | | | controlling agent |
| Hydroxypropyl | / | 71 | / | Release |
| cellulose MF | | | | controlling agent |
| Colloidal silica | 1 | 1 | 1 | Glidant |
| Magnesium stearate | 1 | 1 | 1 | Lubricant |
| Total weight | 200 | 200 | 200 | / |

TABLE 2
Composition of formulation for immediate-release tablet (2.5 mg)

| | Formulation 4 | |
|---|---|---|
| Component | mg/tablet | Function |
| Minoxidil | 2.5 | Active ingredient |
| Mannitol | 63.17 | Filler |
| Microcrystalline cellulose PH102 | 126.33 | Filler |
| Croscarmellose sodium | 6 | Disintegrant |
| Magnesium stearate | 2 | Lubricant |
| Total weight | 200 | / |

The tablet prepared from the immediate-release dosage form disintegrates rapidly within 60 seconds, with a dissolution of 100% in 5 minutes. The immediate-release tablet was prepared by the general mixing and tableting method.

The formulations in Table 1 were prepared as follows:
(1) A prescribed amount of minoxidil and microcrystalline cellulose were mixed in a volume ratio of 1:2, with a rotational speed of 14 rpm for 7 min.
(2) A prescribed amount of mannitol and a remaining microcrystalline cellulose were sieved together through a 35-mesh sieve, and then mixed with a resulting material in step 1, with a rotational speed of 14 rpm for 7 min.
(3) A resulting material in step 2 was subjected to sieving with a granulate machine, where the granulate machine had an aperture of 1016 μm, with a rotational speed of 1750 rpm.
(4) Prescribed amounts of release controlling agent and colloidal silica were subjected to the sieving with the granulate machine in step 3, where the granulate machine had an aperture of 1016 μm, with a rotational speed of 1750 rpm.
(5) A resulting mixture obtained from step 3 and a resulting mixture obtained from step 4 were mixed, with a rotational speed of 14 rpm for 7 min.
(6) Magnesium stearate, sieved through a 60-mesh sieve, was added to a hopper in step 5 for total mixing, with a rotational speed of 14 rpm for 7 min.
(7) Finally, a resulting system was compressed into tablets to obtain the desired tablets.

Test method for release determination: A test sample was taken, and the test was performed according to the USP apparatus 2 method, using 900 mL of pH 7.2 phosphate buffer as a dissolution medium at a rotational speed of 75 rpm. Samples were taken separately at different time points, and filtered through a 0.45 μm filter membrane. A subsequent filtrate was taken as a test solution. An appropriate amount of minoxidil reference substance was taken, and dissolved and diluted with a methanol/dissolution medium (80:20) to approximately 0.002 mg/mL for later use. The ultraviolet absorbance of the test solution was measured at 231 nm, and the dissolution of the sample was calculated.

The in vitro cumulative dissolution of the composition of Formulation 1, Formulation 2, and Formulation 3 is shown in Table 3.

TABLE 3
Cumulative dissolution data for Formulation
1, Formulation 2, and Formulation 3

| | Cumulative dissolution, % | | | | | |
|---|---|---|---|---|---|---|
| | Formulation 1- | | Formulation 2- | | Formulation 3- | |
| Sampling time | HPMC K4M | | HPC | | PEO | |
| point, hour | Mean | RSD % | Mean | RSD % | Mean | RSD % |
| 1 | 19 | 1.2 | 21 | 1.8 | 21 | 1.6 |
| 2 | 30 | 2.1 | 32 | 1.8 | 30 | 2.8 |
| 4 | 48 | 1.8 | 51 | 1.9 | 50 | 1.8 |
| 6 | 60 | 1.0 | 63 | 1.6 | 62 | 1.4 |
| 8 | 70 | 1.5 | 74 | 2.3 | 73 | 2.6 |
| 10 | 78 | 2.5 | 80 | 2.4 | 82 | 2.7 |
| 12 | 84 | 2.2 | 86 | 1.4 | 86 | 1.4 |
| 14 | 89 | 1.1 | 92 | 1.6 | 90 | 1.4 |
| 16 | 93 | 1.0 | 95 | 1.3 | 95 | 1.4 |
| 20 | 97 | 0.7 | 99 | 0.6 | 98 | 0.9 |
| 24 | 99 | 0.5 | 100 | 0.3 | 99 | 0.6 |

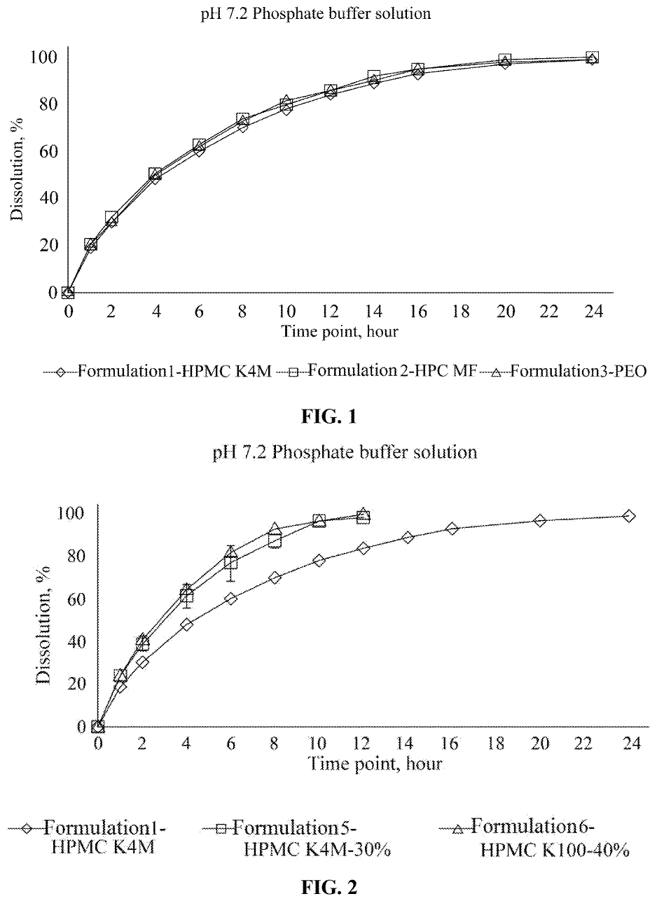

The cumulative dissolution curves of Formulation 1, Formulation 2, and Formulation 3 are shown in FIG. 1 below.

Conclusion: The above data show that the different release controlling agents in Formulations 1, 2, and 3 have similar in vitro dissolution, all achieving a 12-hour sustained-release effect. Next, the inventors investigated the relative bioavailability of each of the above three formulations compared with that of the immediate-release tablet at the same dose based on in vivo PK behavior in dogs.

Study objective: To investigate the pharmacokinetic (PK) characteristics and the relative bioavailability (compared with an immediate-release tablet) of minoxidil sustained-release tablets in beagle dogs at different doses following their single oral administration.

Test design: A single-center, open-label, and single-dose parallel-controlled test was adopted.

Test drugs: Tablets compressed using Formulation 1, Formulation 2, Formulation 3, and Formulation 4, respectively.

Number of subjects: A total of 20 cases, 5 cases/group

Research method: All dogs were fasted for at least 10 h the day before administration, and were administrated minoxidil sustained-release tablets orally at D1 single fasting state, with an administration time fixed at 8:30 am±0.5 h.

Test method: The serum concentration of minoxidil was determined by the LC-MS/MS method.

The test results are as follows:

TABLE 4
Pharmacokinetic parameters at different doses

| | | Cₘₐₓ | AUC₀₋ₜ | AUC_{0-∞} | Relative | |
|---|---|---|---|---|---|---|
| Dose | n | (ng/mL) | (h · ng/mL) | (h · ng/mL) | bioavailability | Tₘₐₓ |
| Formulation 1- | 5 | 3.24 ± 1.23 | 15.67 ± 4.66 | 16.00 ± 4.75 | 65.7% | 1.0 (1.0, 3.0) |
| HPMC K4M | | | | | | |
| Formulation 2- | 5 | 2.716 ± 2.23 | 13.66 ± 4.32 | 13.79 ± 4.01 | 56.6% | 1.5 (0.5, 3) |
| HPC-MF | | | | | | |
| Formulation 3- | 5 | 10.33 ± 1.63 | 19.34 ± 5.32 | 19.77 ± 4.99 | 81.2% | 1.5 (1.0, 2.0) |
| PEO | | | | | | |
| Formulation 4 | 5 | 20.62 ± 6.77 | 24.14 ± 4.57 | 24.34 ± 4.68 | Reference | 0.25 (0.25, 0.25) |
| (immediate- | | | | | | |
| release tablet) | | | | | | |

The pharmacokinetic results show an unexpected finding that the three release controlling agents exhibit identical performance in vitro but show significant differences in animals. The formulation with polyethylene oxide (PEO) as the release controlling agent has the highest relative bioavailability (81.2%), but its maximum plasma concentration is above the target concentration of 10 ng/mL. The formulations with hypromellose (HPMC) and hydroxypropyl cellulose (HPC) as the release controlling agents have relative bioavailabilities of 65.7% and 56.6%, respectively. Therefore, Formulation 1, with a relatively higher bioavailability, is selected for further study.

### Example 2—Investigation of Different Sustained-Release Behaviors

Example 1 demonstrates that hypromellose has good in vivo performance in animals, but its relative bioavailability is low at 65.7%, which is probably due to slow release and poor colonic absorption. To verify this conclusion, the inventors also developed formulations using hypromellose as a sustained-release matrix with faster in vitro release (8-hour sustained-release), and compared the relative bioavailability between the two preparations. The composition of the formulations is shown in Table 5.

TABLE 5
Composition of formulations with different sustained-release behaviors

| | Formulation 1 | | | |
|---|---|---|---|---|
| Component | mg/tablet | Formulation 5 | Formulation 6 | Function |
| Minoxidil | 2.5 | 2.5 | 2.5 | Active ingredient |
| Mannitol 200SD | 20 | 30 | 20 | Filler |
| Microcrystalline | 95.5 | 105.5 | 95.5 | Filler |
| cellulose PH102 | | | | |
| Hypromellose K4M | 80 | 60 | / | Release |
| | | | | controlling agent |
| Hypromellose K100 | / | / | 80 | Release |
| | | | | controlling agent |
| Colloidal silica | 1 | 1 | 1 | Glidant |
| Magnesium stearate | 1 | 1 | 1 | Lubricant |
| Total weight | 200 | 200 | 200 | / |

After the tablets of each formulation were compressed, the cumulative dissolution of the obtained tablets is shown in Table 6.

TABLE 6
Cumulative dissolution data for Formulation
1, Formulation 5, and Formulation 6

| | Cumulative dissolution, % | | | | | |
|---|---|---|---|---|---|---|
| | Formulation 1- | | Formulation 5- | | Formulation 6- | |
| | HPMC | | HPMC | | HPMC | |
| Sampling time | K4M-40% | | K4M-30% | | K100-40% | |
| point, hour | Mean | RSD % | Mean | RSD % | Mean | RSD % |
| 1 | 19 | 1.2 | 24 | 1.8 | 25 | 1.6 |
| 2 | 30 | 2.1 | 39 | 10.5 | 41 | 2.8 |
| 4 | 48 | 1.8 | 61 | 7.7 | 65 | 2.1 |
| 6 | 60 | 1.0 | 77 | 8.2 | 82 | 1.2 |
| 8 | 70 | 1.5 | 88 | 9.9 | 93 | 2.1 |
| 10 | 78 | 2.5 | 97 | 3.5 | 97 | 0.6 |
| 12 | 84 | 2.2 | 99 | 1.4 | 101 | 0.6 |
| 14 | 89 | 1.1 | / | / | / | / |
| 16 | 93 | 1.0 | / | / | / | / |
| 20 | 97 | 0.7 | / | / | / | / |
| 24 | 99 | 0.5 | / | / | / | / |

The cumulative dissolution curves of Formulation 1, Formulation 5, and Formulation 6 are shown in FIG. 2 below.

Conclusion: In FIG. 2, Formulation 5 can achieve an in vitro 8-hour sustained release by reducing the proportion of hypromellose K4M, but exhibits a large dissolution difference with a relative standard deviation greater than 5% (Formulation 5). This difference can easily lead to large fluctuations in in vivo plasma concentration, affecting safety. The formulation using hypromellose K100 with relatively low viscosity not only achieves an in vitro 8-hour sustained release but also results in a small dissolution difference (Formulation 6). To compare the relative bioavailability of fast release (8-hour sustained-release) versus slow release (12-hour sustained-release), the following human test was performed.

Study object: To investigate the relative bioavailability of a low-viscosity minoxidil sustained-release tablet versus a high-viscosity minoxidil sustained-release tablet in healthy male subjects following their single oral administration under fasting conditions.

Test design: A single-center, open-label, single-dose double crossover test was adopted.

Test drugs: Tablets compressed using Formulation 1 and Formulation 6, respectively Number of subjects: A total of 6 healthy volunteers

Study design: The night before administration in each period, subjects were fasted overnight for at least 10 hours. Under fasting conditions and maintaining an upright upper body position, the subjects took one minoxidil sustained-release tablet (strength: 1.8 mg) or one minoxidil immediate-release tablet (strength: 2.5 mg) with 240 mL of warm water, with the following administration solutions:

TABLE 7
Route of administration

| | | Number | | |
|---|---|---|---|---|
| Test status | Group | of cases | Period 1 | Period 2 |
| Fasting test | Group A | 3 | High-viscosity minoxidil | Low-viscosity minoxidil |
| | | | sustained-release tablet | sustained-release tablet |
| | | | (Formulation 1) | (Formulation 6) |
| | Group B | 3 | Low-viscosity minoxidil | High-viscosity minoxidil |
| | | | sustained-release tablet | sustained-release tablet |
| | | | (Formulation 6) | (Formulation 1) |

The test results are as follows.

TABLE 8
PK parameters

| | | Cₘₐₓ | AUC₀₋ₜ | AUC_{0-∞} | Relative | |
|---|---|---|---|---|---|---|
| Dose | n | (ng/mL) | (h · ng/mL) | (h · ng/mL) | bioavailability | Tₘₐₓ |
| High-viscosity minoxidil | 6 | 4.83 ± 2.13 | 23.05 ± 4.49 | 23.77 ± 4.64 | Reference | 1.0 (0.5, 3.0) |
| sustained-release tablet | | | | | | |
| (Formulation 1) | | | | | | |
| Low-viscosity minoxidil | 6 | 6.33 ± 2.23 | 26.77 ± 8.66 | 27.15 ± 8.75 | 114.2% | 1.5 (0.5, 3.0) |
| sustained-release tablet | | | | | | |
| (Formulation 6) | | | | | | |

Conclusion: Compared with the high-viscosity minoxidil sustained-release tablet (Formulation 1), the low-viscosity minoxidil sustained-release tablet (Formulation 6) shows a 14% increase in relative bioavailability, and its plasma concentration is lower than the target limit of 10 ng/ml. This indicates that the bioavailability of Formulation 6 compared with that of the immediate-release tablet at the same dose is approximately 80%.

### Example 3—Stability Study of Formulation 6

Formulation 6 is an optimized formulation, which not only has good bioavailability but also has a plasma concentration lower than 10 ng/mL. The inventors investigated Formulation 6 in accelerated and long-term stability studies.

The dissolution data for Formulation 6 after 6 months under accelerated conditions and after 6 months under long-term conditions are shown in the table below.

TABLE 9
Cumulative dissolution data for Formulation
6 under different conditions

| | Cumulative dissolution, % | | | | | |
|---|---|---|---|---|---|---|
| | | | Formulation 6- | | Formulation 6- | |
| | Formulation 6- | | 40° C./75% | | 25° C./60%- | |
| Sampling time | 0 day | | RH 6 months | | 12 months | |
| point, hour | Mean | Mean | Mean | RSD % | Mean | RSD % |
| 1 | 25 | 1.6 | 18 | 2.8 | 19 | 4.6 |
| 2 | 41 | 2.8 | 34 | 11.6 | 35 | 8.5 |
| 4 | 65 | 2.1 | 58 | 6.4 | 60 | 12.5 |
| 6 | 82 | 1.2 | 73 | 7.4 | 75 | 10.4 |
| 8 | 93 | 2.1 | 83 | 8.4 | 86 | 8.7 |
| 10 | 97 | 0.6 | 89 | 6.5 | 92 | 4.8 |
| 12 | 101 | 0.6 | 93 | 5.4 | 95 | 3.6 |

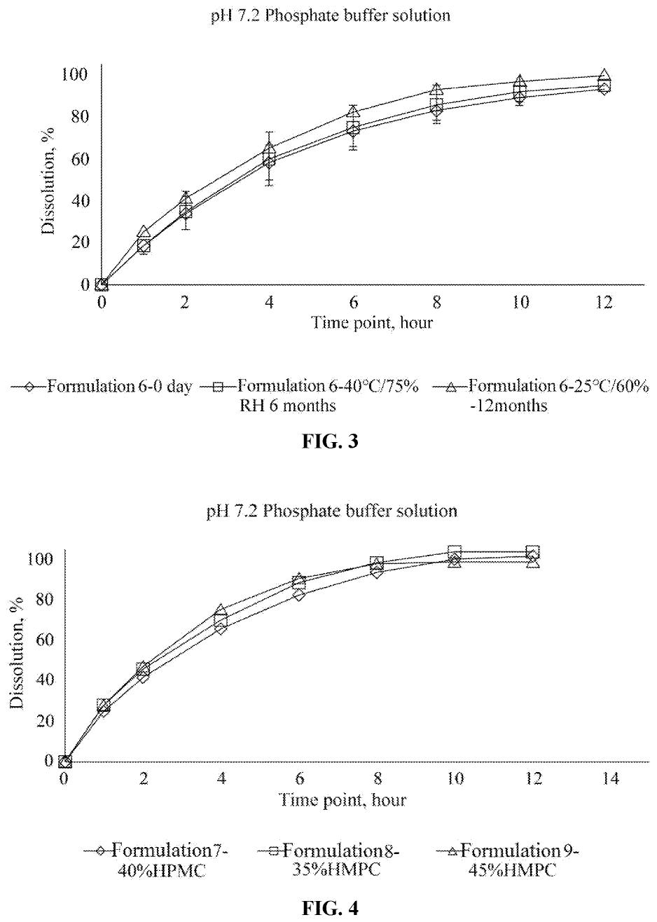

The dissolution curves are shown in FIG. 3 below.

Conclusion: The dissolution of Formulation 6 is significantly reduced after 6 months under accelerated conditions and after 12 months under long-term conditions. Moreover, the relative standard deviation (RSD) of dissolution is greater than 5%. Such changes in dissolution may lead to alterations in in vivo behavior, posing an extremely high risk.

Based on the experience, hypromellose in the formulation will not cause changes in the dissolution curve due to its sufficient sustained-release capacity. It is speculated that the interaction between the filler and the drug leads to the alterations in the crystal form or particle size of minoxidil, which in turn causes the alterations in the dissolution. Based on the above speculation, the inventors investigated the compatibility between minoxidil and different fillers.

The samples were divided into four groups in parallel and placed under the conditions of freeze-sealing (−20° C.), high temperature-sealing (60° C.), light-resistant sealing, and high humidity (92.5% RH) opening, respectively. The test periods were 15 days and 30 days. Among these, the freeze-sealing (−20° C.) served as a control group to facilitate the identification of factors affecting the occurrence of degradation. Under the conditions of high temperature and high humidity, minoxidil and excipients were weighed and placed in brown bottles, respectively. For the light exposure condition, they were placed in transparent glass bottles. After thorough mixing, the samples were placed separately according to the four conditions designed above. The specific types of excipients involved, mixing ratios, test conditions, and results are shown in the table below.

TABLE 10
Excipient compatibility (binary mixture)

| | | | | | Related substances (%) | |
|---|---|---|---|---|---|---|
| | | | | | Unknown | |
| | Active | | | Content | single | Total |
| Excipient | Ingredient:Excipient | Condition and time | Appearance | (%) | impurity | impurities |
| Minoxidil | / | Freeze-sealing, 0 day | White powder | 99.5% | ND | ND |
| | | 60° C.-sealing, 15 days | White powder | 96.4% | ND | ND |
| | | 60° C.-sealing, 30 days | White powder | 101.1% | ND | ND |
| | | 92.5% RH-sealing, 15 days | White powder | 98.5% | ND | ND |
| | | 92.5% RH-sealing, 30 days | White powder | 101.3% | ND | ND |
| | | Light-resistant sealing, 15 days | White powder | 98.4% | ND | <LOQ |
| | | Light-resistant sealing, 30 days | White powder | 101.2% | ND | ND |
| Mannitol | 1:25 | Freeze-sealing, 0 day | White powder | 98.5% | | |
| | | 60° C.-sealing, 15 days | Pale yellow | 97.1% | 0.23% | 0.23% |
| | | | powder | | | |
| | | 60° C.-sealing, 30 days | Pale yellow | 96.4% | 1.54% | 1.54% |
| | | | powder | | | |
| | | 92.5% RH-sealing, 15 days | Pale yellow | 99.0% | ND | <LOQ |
| | | | powder | | | |
| | | 92.5% RH-sealing, 30 days | Pale yellow | 100.9% | ND | ND |
| | | | powder | | | |
| | | Light-resistant sealing, 15 days | White powder | 97.9% | ND | ND |
| | | Light-resistant sealing, 30 days | White powder | 101.5% | ND | <LOQ |
| Lactose | 1:25 | Freeze-sealing, 0 day | White powder | 100.3% | ND | ND |
| | | 60° C.-sealing, 15 days | White powder | 101.6% | ND | ND |
| | | 60° C.-sealing, 30 days | White powder | 98.6% | ND | <LOQ |
| | | 92.5% RH-sealing, 15 days | White powder | 100.8% | ND | ND |
| | | 92.5% RH-sealing, 30 days | White powder | 96.4% | ND | ND |
| | | Light-resistant sealing, 15 days | White powder | 100.1% | ND | ND |
| | | Light-resistant sealing, 30 days | White powder | 98.9% | ND | ND |
| Xylitol | 1:25 | Freeze-sealing, 0 day | White powder | 100.3% | ND | ND |
| | | 60° C.-sealing, 15 days | White powder | 101.6% | ND | ND |
| | | 60° C.-sealing, 30 days | White powder | 98.6% | ND | ND |
| | | 92.5% RH-sealing, 15 days | White powder | 100.8% | ND | ND |
| | | 92.5% RH-sealing, 30 days | White powder | 96.4% | ND | ND |
| | | Light-resistant sealing, 15 days | White powder | 100.1% | ND | ND |
| | | Light-resistant sealing, 30 days | White powder | 98.9% | ND | ND |
| Calcium | 1:25 | Freeze-sealing, 0 day | White powder | 97.9% | ND | ND |
| hydrogen | | 60° C.-sealing, 15 days | White powder | 100.5% | ND | ND |
| phosphate | | 60° C.-sealing, 30 days | White powder | 98.5% | <LOQ | <LOQ |
| | | 92.5% RH-sealing, 15 days | White powder | 100.8% | ND | ND |
| | | 92.5% RH-sealing, 30 days | White powder | 96.8% | ND | ND |
| | | Light-resistant sealing, 15 days | White powder | 100.6% | ND | ND |
| | | Light-resistant sealing, 30 days | White powder | 100.3% | ND | ND |
| Microcrystalline | 1:25 | Freeze-sealing, 0 day | White powder | 98.8% | ND | ND |
| cellulose | | 60° C.-sealing, 15 days | White powder | 97.5% | ND | ND |
| | | 60° C.-sealing, 30 days | White powder | 101.3% | <LOQ | <LOQ |
| | | 92.5% RH-sealing, 15 days | White powder | 99.1% | ND | ND |
| | | 92.5% RH-sealing, 30 days | White powder | 101.0% | <LOQ | <LOQ |
| | | Light-resistant sealing, 15 days | White powder | 100.5% | ND | ND |
| | | Light-resistant sealing, 30 days | White powder | 101.1% | ND | ND |

The compatibility results show that minoxidil and mannitol generate impurities at high temperature but they are stable under both high-humidity and light-exposure conditions. Minoxidil is stable under high-temperature, high-humidity, and light-exposure conditions when combined with calcium hydrogen phosphate, microcrystalline cellulose, xylitol, and lactose.

Therefore, it can be inferred that the decrease in content during the stability study of the minoxidil sustained-release tablet is caused by mannitol. Consequently, the inventors replaced the filler mannitol with lactose, investigated the in vitro dissolution, and placed it for stability study.

Although Formulation 6 has been screened out to achieve the development objects of the present disclosure, its stability was poor. It is inferred that simply switching the filler from mannitol to lactose with similar properties will not affect in vivo absorption. According to literature review, the topical minoxidil solution has a systemic exposure of 18.71 ng·hr/mL, while the 2.5 mg minoxidil XR tablet (Formulation 6) has a systemic exposure of 26.77 ng·hr/mL. Based on dose correction, the dose need to be reduced to achieve a systemic exposure of 18.71 ng·hr/mL in vivo, and can be calculated as: 18.71 ng·hr/mL×2.5 mg/26.77 ng·hr/mL=1.75 mg. Therefore, it can be inferred that a 1.8 mg sustained-release tablet can achieve a systemic exposure equivalent to that of the topical minoxidil solution, with a further reduction in the maximum plasma concentration, thereby significantly improving safety.

The next step was to verify the above inferences.

### Example 4—Relative Bioavailability of 1.8 mg Sustained-Release Tablet and Commercially Available Minoxidil Topical Solution

Based on Formulation 6, Formulation 7 containing lactose was designed. The composition of Formulation 7 is shown in Table 11.

TABLE 11
Composition of formulation for 1.8 mg sustained-release tablet

| | Formulation 7 | |
|---|---|---|
| Component | mg/tablet | Function |
| Minoxidil | 1.8 | Active ingredient |
| Lactose | 20 | Filler |
| Microcrystalline | 96.2 | Filler |
| cellulose PH102 | | |
| Hypromellose K100 | 80 | Release controlling |
| | | agent |
| Colloidal silica | 1 | Glidant |
| Magnesium stearate | 1 | Lubricant |
| Total weight | 200 | / |

The pharmacokinetic characteristics of Formulation 7 and commercially available minoxidil topical solution were investigated.

Study object: To investigate the pharmacokinetic (PK) characteristics and the relative bioavailability (compared with multiple topical administrations of 5% minoxidil topical solution (Rogaine®)) of 1.8 mg minoxidil sustained-release tablet in healthy male subjects following its single oral administration.

Test design: A single-center, open-label, single-dose and multiple-dose study was adopted.

Test drug: Test preparation: Tablet compressed using Formulation 7

Reference: Minoxidil solution (trade name: Rogain®); Specification: 5%; Batch No.: 3432CP; Valid until: November 2025; Package: 60 mL/bottle; Storage conditions: Store at controlled room temperature 20° C. to 25° C. or below. Manufacturer: JOHNSON & JOHNSON CONSUMER INC.

Number of subjects: A total of 6 healthy volunteers

Research method: Single oral administration: The subjects were fasted for at least 10 h the day before administration, and all subjects were administrated 1.8 mg minoxidil sustained-release tablets orally at D1 single fasting state, with an administration time fixed at 8:30 am±0.5 h. On D1, the subjects needed to maintain an upright upper body position within 4 h after administration.

Multiple topical administrations: D4 to D8 were multiple topical administrations, twice daily (at 12-hour intervals). Using a dropper, 1 mL of 5% Minoxidil Topical Solution (Rogaine®) was drawn up, applied to the scalp, and massaged into the scalp until absorbed. The daily administration times were fixed at 8:30 am±0.5 h and 8:30 μm±0.5 h, respectively. On D9, a single morning dose was administered: using a dropper, 1 mL of 5% Minoxidil Topical Solution (Rogaine®) was drawn up, applied to the scalp, and massaged into the scalp until absorbed. The administration time was 8:30 am±0.5 h. From D4 to D9, contact with the head in any way was avoided for 4 h after administration.

Washout period: 3 days

Test method: The serum concentration of minoxidil was determined by the LC-MS/MS method.

The test results are as follows:

TABLE 12
Pharmacokinetic parameters of 1.8 mg sustained-
release tablet and minoxidil solution

| | Formulation 7 | | Reference |
|---|---|---|---|
| | Arithmetic | | Arithmetic |
| Parameter | mean ± SD | Parameter | mean ± SD |
| (unit) | (N = 6) | (unit) | (N = 6) |
| Tmax (h) | 1.06 ± 0.52 | Tss, max (h) | 2.63 ± 1.49 |
| Cmax (ng/mL) | 4.23 ± 1.52 | Css, max (ng/mL) | 2.20 ± 0.591 |
| AUC0-24 | 19.44 ± 5.37 | AUCss, 0-24 | 18.96 ± 5.621 |
| (h · ng/mL) | | (h · ng/mL) | |

Compared with the minoxidil topical solution, the relative bioavailability is: 102%

Conclusion: The test results demonstrate that the minoxidil sustained-release tablet (Formulation 7) and the reference have similar systemic exposure. The relative bioavailability is 102%, and the maximum plasma concentration is lower than the target limit concentration of 10 ng/mL. Therefore, Formulation 7 is the target formulation determined by the present disclosure.

### Example 5—Study on Different Hypromellose K100 Levels

Hypromellose, as a sustained-release matrix, directly affects the dissolution behavior of minoxidil sustained-release tablets. To determine an appropriate hypromellose level, the inventors investigated the hypromellose levels. The composition of the formulations is shown in the table below.

TABLE 13
Composition of formulations with different hypromellose levels

| | Formulation 7 | Formulation 8 | Formulation 9 | |
|---|---|---|---|---|
| | 40% HPMC | 35% HPMC | 45% HPMC | |
| Component | mg/tablet | | | Function |
| Minoxidil | 1.8 | 1.8 | 1.8 | Active ingredient |
| Lactose | 20 | 25 | 15 | Filler |
| Microcrystalline | 96.2 | 101.2 | 91.2 | Filler |
| cellulose PH102 | | | | |
| Hypromellose | 80 | 70 | 90 | Release controlling |
| K100 | | | | agent |
| Colloidal silica | 1 | 1 | 1 | Glidant |
| Magnesium | 1 | 1 | 1 | Lubricant |
| stearate | | | | |
| Total weight | 200 | 200 | 200 | / |

The tablets compressed using Formulation 7, Formulation 8, and Formulation 9 were subjected to a dissolution study. The data are shown in the table below.

TABLE 14
Cumulative dissolution data at different hypromellose levels

| | Cumulative dissolution, % | | | | | |
|---|---|---|---|---|---|---|
| | Formulation 7 | | Formulation 8 | | Formulation 9 | |
| Sampling time | 40% HPMC | | 35% HPMC | | 45% HPMC | |
| point, hour | Mean | Mean | Mean | RSD % | Mean | RSD % |
| 1 | 26 | 2.9 | 27 | 1.6 | 28 | 2.3 |
| 2 | 42 | 2.1 | 45 | 1.8 | 47 | 2.1 |
| 4 | 66 | 2.1 | 70 | 1.1 | 75 | 2.8 |
| 6 | 83 | 2.3 | 88 | 1.2 | 90 | 1.1 |
| 8 | 95 | 1.5 | 98 | 1.1 | 97 | 1.3 |
| 10 | 100 | 1.4 | 103 | 1.0 | 98 | 1.0 |
| 12 | 101 | 0.2 | 103 | 1.6 | 98 | 0.9 |

| | | | |
|---|---|---|---|
| F2 | Control | 66 | 70 |

The higher F2 is, the more similar the curves. Generally, if F2 is greater than 50, it can be considered that the curves are similar.

The dissolution curves are shown in FIG. 4 below.

Conclusion: As can be seen from FIG. 4, compared with the target formulation (Formulation 7), when the proportion of hypromellose in the formulation is 35% to 45%, the similarity factor f2 is greater than 60. Therefore, the proportion of hypromellose in the formulation is selected to be 35% to 45%.

### Example 6—Study on Microcrystalline Cellulose/Lactose Ratios

Microcrystalline cellulose is an insoluble excipient, while lactose is a readily soluble excipient. Different ratios of the microcrystalline cellulose to the lactose may affect the dissolution of tablets. To screen for an optimal microcrystalline cellulose/lactose ratio, the inventors designed four levels of microcrystalline cellulose/lactose ratios for investigation, i.e., 4.81:1, 3:1, 2:1, and 1:1. The composition of the formulations is shown in the table below.

TABLE 15
Composition of formulations with different microcrystalline cellulose/lactose ratios

| | Formulation 7 | Formulation 9 | Formulation 10 | Formulation 11 | |
|---|---|---|---|---|---|
| | Microcrystalline | Microcrystalline | Microcrystalline | Microcrystalline | |
| | cellulose/ | cellulose/ | cellulose/ | cellulose/ | |
| | lactose = 4.81:1 | lactose = 3:1 | lactose = 2:1 | lactose = 1:1 | |
| Component | mg/tablet | | | | Function |
| Minoxidil | 1.8 | 1.8 | 1.8 | 1.8 | Active |
| | | | | | ingredient |
| Lactose F100 | 20 | 29.01 | 38.73 | 58.1 | Filler |
| Microcrystalline | 96.2 | 87.19 | 77.47 | 58.1 | Filler |
| cellulose PH102 | | | | | |
| Hypromellose K100 | 80 | 80 | 80 | 80 | Release |
| | | | | | controlling |
| | | | | | agent |
| Colloidal silica | 1 | 1 | 1 | 1 | Glidant |
| Magnesium stearate | 1 | 1 | 1 | 1 | Lubricant |
| Total weight | 200 | 200 | 200 | 200 | / |

### Preparation Process 2 was Used for Preparation

Tablet compression situation: Formulation 11 could be compressed up to a maximum of 120 N and failed to compress to the target hardness of 150 N. This may be caused by the reduced microcrystalline cellulose content and poor compressibility.

TABLE 16
Cumulative dissolution data of Formulation 7 and Formulation 9 to Formulation 11

| | Cumulative dissolution, % | | | | | | | |
|---|---|---|---|---|---|---|---|---|
| | Formulation 7 | | Formulation 9 | | Formulation 10 | | Formulation 11 | |
| Sampling | Microcrystalline | | Microcrystalline | | Microcrystalline | | Microcrystalline | |
| time point, | cellulose/lactose = 4.81:1 | | cellulose/lactose = 3:1 | | cellulose/lactose = 2:1 | | cellulose/lactose = 1:1 | |
| hour | Mean | Mean | Mean | RSD % | Mean | RSD % | Mean | RSD % |
| 1 | 25 | 2.9 | 25 | 4.3 | 26 | 4.3 | 30 | 1.3 |
| 2 | 41 | 2.1 | 43 | 2.1 | 45 | 2.1 | 50 | 1.2 |
| 4 | 65 | 2.1 | 67 | 2.8 | 69 | 2.8 | 79 | 1.1 |
| 6 | 82 | 2.3 | 85 | 1.1 | 79 | 1.1 | 94 | 1.3 |
| 8 | 93 | 1.5 | 95 | 1.3 | 97 | 1.3 | 99 | 0.9 |
| 10 | 100 | 1.4 | 99 | 1 | 98 | 1 | 101 | 1 |
| 12 | 101 | 0.2 | 99 | 0.9 | 98 | 0.9 | 101 | 0.9 |
| F2 relative to | Control | | 82 | | 74 | | 70 | |
| Formulation 6- | | | | | | | | |

The higher F2 is, the more similar the curves. Generally, if F2 is greater than 50, it can be considered that the curves are similar.

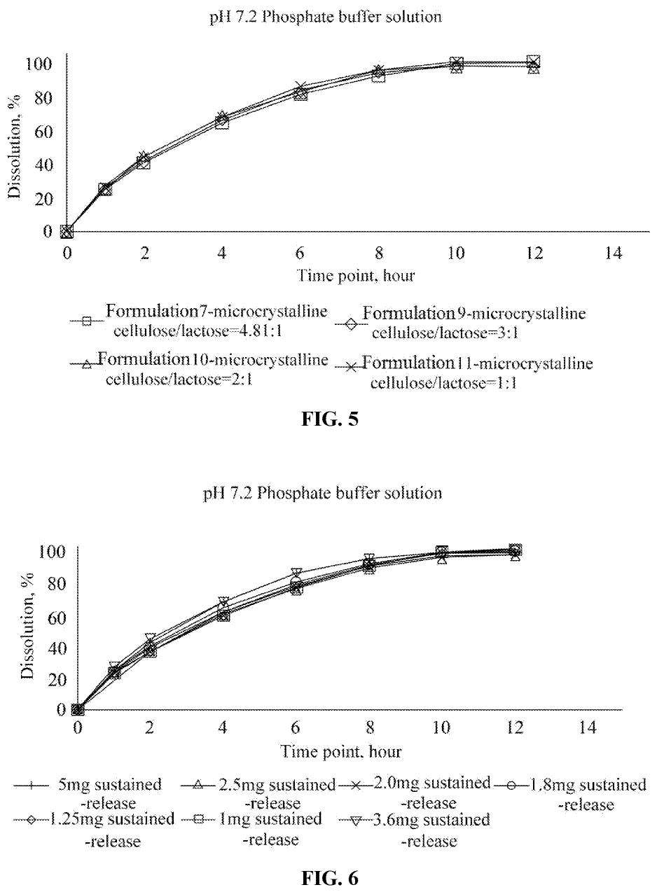

The dissolution curves are shown in FIG. 5 below.

Conclusion: In FIG. 5, Formulation 7, Formulation 9, Formulation 10, and Formulation 11 are investigated separately. It can be seen that when the microcrystalline cellulose/lactose ratio is 4.81:1 to 1:1, the dissolution of the minoxidil sustained-release tablets is similar. However, as the microcrystalline cellulose content decreases, the compressibility declines. Therefore, the microcrystalline cellulose/lactose ratio is preferably 4.81:1 to 2:1.

### Example 7—Effect of Different Processes on Content Uniformity of Tablets

Due to the low proportion of the minoxidil in the formulation, the content uniformity of the tablet has a relatively high risk to tablet quality. To screen for a suitable process, using Formulation 7 as the target formulation, the effect of the following processes on the content uniformity of the tablet was investigated.

### Process 1:

(1) Prescribed amounts of minoxidil, lactose, microcrystalline cellulose, hypromellose, and colloidal silica were pre-mixed, with a rotational speed of 14 rpm for 14 min.
(2) Magnesium stearate, sieved through a 60-mesh sieve, was added to a resulting material for total mixing, with a rotational speed of 14 rpm for 7 min.
(3) A resulting system was compressed into target tablet with a weight of 200 mg, and a hardness of 150 N.

### Process 2:

(1) A prescribed amount of minoxidil and microcrystalline cellulose were pre-mixed in a volume ratio of 1:2, with a rotational speed of 14 rpm for 7 min.
(2) A prescribed amount of lactose and a remaining microcrystalline cellulose were sieved together through a 35-mesh sieve, and then mixed with a resulting material in step (1), with a rotational speed of 14 rpm for 7 min.
(3) A resulting material in step (2) was subjected to sieving with a granulate machine, where the granulate machine had an aperture of 1016 μm, with a rotational speed of 1750 rpm.
(4) Prescribed amounts of hypromellose and colloidal silica were subjected to the granulate machine sieving in step (3), where the granulate machine had an aperture of 1016 μm, with a rotational speed of 1750 rpm.
(5) A resulting mixture obtained from step (3) and a resulting mixture obtained from step (4) were mixed, with a rotational speed of 14 rpm for 7 min.
(6) Magnesium stearate, sieved through a 60-mesh sieve, was added to a hopper in step (5) for total mixing, with a rotational speed of 14 rpm for 7 min.
(7) A resulting system was compressed into target tablet with a weight of 200 mg and a hardness of 150 N.

### Process 3:

(1) A prescribed amount of minoxidil and microcrystalline cellulose were pre-mixed in a volume ratio of 1:5, with a rotational speed of 14 rpm for 7 min.
(2) A prescribed amount of lactose and a remaining microcrystalline cellulose were sieved together through a 35-mesh sieve, and then mixed with a resulting material in step (1), with a rotational speed of 14 rpm for 7 min.
(3) A resulting material from step (2) was subjected to sieving with a granulate machine, where the granulate machine had an aperture of 1016 μm, with a rotational speed of 1750 rpm.
(4) Prescribed amounts of hypromellose and colloidal silica were subjected to the granulate machine sieving in step (3), where the granulate machine had an aperture of 1016 μm, with a rotational speed of 1750 rpm.
(5) A resulting mixture obtained from step (3) and a resulting mixture obtained from step (4) were mixed, with a rotational speed of 14 rpm for 7 min.
(6) Magnesium stearate, sieved through a 60-mesh sieve, was added to a hopper in step (5) for total mixing, with a rotational speed of 14 rpm for 7 min.
(7) A resulting system was compressed into target tablet with a weight of 200 mg and a hardness of 150 N.

### Process 4:

(1) A prescribed amount of minoxidil and microcrystalline cellulose were pre-mixed in a volume ratio of 1:10, with a rotational speed of 14 rpm for 7 min.
(2) A prescribed amount of lactose and a remaining microcrystalline cellulose were sieved together through a 35-mesh sieve, and then mixed with a resulting material in step (1), with a rotational speed of 14 rpm for 7 min.
(3) A resulting material in step (2) was subjected to sieving with a granulate machine, where the granulate machine had an aperture of 1016 μm, with a rotational speed of 1750 rpm.
(4) Prescribed amounts of hypromellose and colloidal silica were subjected to the granulate machine sieving in step (3), where the granulate machine had an aperture of 1016 μm, with a rotational speed of 1750 rpm.
(5) A resulting mixture obtained from step (3) and a resulting mixture obtained from step (4) were mixed, with a rotational speed of 14 rpm for 7 min.
(6) Magnesium stearate, sieved through a 60-mesh sieve, was added to a hopper in step (5) for total mixing, with a rotational speed of 14 rpm for 7 min.
(7) A resulting system was compressed into target tablet with a weight of 200 mg and a hardness of 150 N.

The tablets were prepared by Process 1, Process 2, Process 3, and Process 4. The content uniformity of the tablets is shown in the table below.

TABLE 17
Summary of content uniformity of tablets prepared by different processes

| | | Process 2 | Process 3 | Process 4 |
|---|---|---|---|---|
| | | Premixing: | Premixing: | Premixing: |
| | Process 1 | Minoxidil/ | Minoxidil/ | Minoxidil/ |
| | No | microcrystalline | microcrystalline | microcrystalline |
| Tablet | premixing | cellulose = 1:2 | cellulose = 1:5 | cellulose = 1:10 |
| 1 | 98.4% | 99.6% | 97.9% | 98.7% |
| 2 | 92.5% | 99.7% | 98.8% | 95.1% |
| 3 | 97.6% | 100.2% | 98.1% | 97.4% |
| 4 | 108.6% | 98.2% | 98.7% | 104.3% |
| 5 | 104.3% | 97.9% | 96.7% | 105.2% |
| 6 | 97.5% | 100.6% | 97.5% | 95.3% |
| 7 | 99.7% | 101.4% | 98.9% | 97.3% |
| 8 | 89.2% | 101.2% | 99.2% | 94.5% |
| 9 | 94.1% | 99.8% | 97.8% | 96.3% |
| 10 | 96.3% | 98.7% | 99.1% | 97.8% |
| Mean | 97.8% | 98.7% | 98.3% | 98.2% |
| Content uniformity | 14.77 | 2.88 | 3.47 | 9.96 |
| (A + 2.2S) limit <15.0 | | | | |

The above data show that with Process 1, the content uniformity deviation of tablets is large, and the AV value for content uniformity is 14.77, almost reaching disqualification. Premixing the minoxidil with the microcrystalline cellulose results in acceptable tablet content uniformity for all processes. However, when the premixing ratio of the minoxidil to the microcrystalline cellulose is 1:10, although the content uniformity meets the limit requirement, the content uniformity deviation is still large. Therefore, the ratio of the minoxidil to the microcrystalline cellulose is selected to be 1:2 to 1:5.

### Example 8—Stability Study

Stability test results: The tablets compressed using Formulation 7, using commercial packaging, were placed under accelerated conditions (40° C./75% RH) for 1 month, 3 months, and 6 months, respectively, and the appearance, content, related substances, and release of the tablets were determined. The results are shown in the table below.

TABLE 18
Stability results of target formulation under accelerated (40° C./75% RH) conditions

| Sample | Placement | | | | | |
|---|---|---|---|---|---|---|
| information | condition | | Appearance | Content, % | Related substances, % | Release, % |
| Formulation 7 | 0 | day | The product is | 99.2 | Unknown single | 1 h: 24.7 |
| (1.8 mg) | | | a white, round | | impurity: ND | 4 h: 64.6 |
| | | | tablet | | Total impurities: ND | 8 h: 92.7 |
| | 1 | month | The product is | 99.5 | Unknown single | 1 h: 25.5 |
| | | | a white, round | | impurity: ND | 4 h: 66.4 |
| | | | tablet | | Total impurities: ND | 8 h: 95.8 |
| | 3 | months | The product is | 99.3 | Unknown single | 1 h: 25.6 |
| | | | a white, round | | impurity: ND | 4 h: 67.8 |
| | | | tablet | | Total impurities: ND | 8 h: 94.9 |
| | 6 | months | The product is | 100.2 | Unknown single | 1 h: 25.6 |
| | | | a white, round | | impurity: ND | 4 h: 68.1 |
| | | | tablet | | Total impurities: ND | 8 h: 95.2 |

Conclusion: The above stability results indicate that after placement for 6 months under accelerated conditions, the target formulation shows essentially no changes in appearance, content, related substances, and release, demonstrating that the formulation is stable.

### Example 9—Composition and In Vitro Dissolution Curves of Formulations at Different Doses

TABLE 19
Composition of formulations at different doses

| | 5 mg | 3.6 mg | 3.2 mg | 2.5 mg | 2 mg | 1.8 mg | 1.25 mg | | |
|---|---|---|---|---|---|---|---|---|---|
| Component | mg/tablet | | | | | | | 1 mg | Function |
| Minoxidil | 5 | 3.6 | 3.2 | 2.5 | 2 | 1.8 | 1.25 | 1.0 | Active ingredient |
| Lactose F100 | 19.5 | 19.5 | 19.5 | 20 | 20 | 20 | 20.15 | 20.15 | Filler |
| Microcrystalline | 93.5 | 94.9 | 95.3 | 95.5 | 96 | 96.2 | 96.6 | 96.85 | Filler |
| cellulose PH102 | | | | | | | | | |
| Hypromellose | 80 | 80 | 80 | 80 | 80 | 80 | 80 | 80 | Release controlling |
| K100 | | | | | | | | | agent |
| Colloidal silica | 1 | 1 | 1 | 1 | 1 | 1 | 1 | 1 | Glidant |
| Magnesium | 1 | 1 | 1 | 1 | 1 | 1 | 1 | 1 | Lubricant |
| stearate | | | | | | | | | |
| Total weight | 200 | 200 | 200 | 200 | 200 | 200 | 200 | 200 | / |

The preparation method was the same as that of Preparation process 2.

The dissolution results for the above different doses are shown in FIG. 6 below. It can be seen from FIG. 6 that the in vitro dissolution of each formulation exhibits a similar dissolution curve. According to the principle of identical formulation, the in vivo bioavailability of the formulations is similar.

What is claimed is:

1. A solid pharmaceutical composition, comprising:
a) a therapeutically effective amount of minoxidil,
b) a pharmaceutically acceptable release controlling agent,
c) a lubricant,
d) a filler, and
e) a glidant, wherein
the therapeutically effective amount is in a range of 1 mg to 10 mg; and
the solid pharmaceutical composition exhibits the following in vitro release profile when tested using a paddle method at a rotational speed of 75 rpm and in 900 mL of a phosphate buffer solution with a pH of 7.2:
at 1 hour, 15% to 30% of the minoxidil is released;
at 4 hours, 55% to 75% of the minoxidil is released; and
at 8 hours, more than 80% of the minoxidil is released.

2. The solid pharmaceutical composition of claim 1, wherein the therapeutically effective amount is in a range of 2 mg to 8 mg.

3. The solid pharmaceutical composition of claim 1, wherein the therapeutically effective amount is 3.2 mg twice daily.

4. The solid pharmaceutical composition of claim 1, wherein the solid pharmaceutical composition is in a form of a tablet, a capsule, or a suspension.

5. The solid pharmaceutical composition of claim 1, wherein the solid pharmaceutical composition consists of: in percentage by weight, 0.1%-5% of the minoxidil, 30%-50% of the pharmaceutically acceptable release controlling agent, 0.1%-2% of the glidant, 0.1%-2% of the lubricant, and a remaining amount of the filler.

6. The solid pharmaceutical composition of claim 1, wherein the solid pharmaceutical composition consists of: in percentage by weight, 0.5%-2.5% of the minoxidil, 35%-45% of the pharmaceutically acceptable release controlling agent, 0.1%-1% of the glidant, 0.1%-1% of the lubricant, and the remaining amount of the filler.

7. The solid pharmaceutical composition of claim 1, wherein the filler contains a hydrophobic component and a hydrophilic component.

8. The solid pharmaceutical composition of claim 7, wherein the hydrophobic component is one or more selected from the group consisting of microcrystalline cellulose and calcium hydrogen phosphate.

9. The solid pharmaceutical composition of claim 7, wherein the hydrophilic component is one or more selected from the group consisting of lactose and xylitol.

10. The solid pharmaceutical composition of claim 1, wherein the pharmaceutically acceptable release controlling agent is one or more selected from the group consisting of hypromellose, hydroxypropyl cellulose, ethyl cellulose, polyethylene oxide, carbomers and methacrylic acid copolymer.

11. The solid pharmaceutical composition of claim 10, wherein the pharmaceutically acceptable release controlling agent is the hypromellose.

12. The solid pharmaceutical composition of claim 1, wherein the glidant is one or more selected from the group consisting of colloidal silica and talc.

13. The solid pharmaceutical composition of claim 1, wherein the lubricant is one or more selected from the group consisting of magnesium stearate and sodium stearyl fumarate.

14. The solid pharmaceutical composition of claim 1, wherein the filler is a combination of microcrystalline cellulose and lactose in a weight ratio of 1-5:1.

15. The solid pharmaceutical composition of claim 1, wherein the filler is a combination of microcrystalline cellulose and lactose in a weight ratio of 2-4.81:1.

16. The solid pharmaceutical composition of claim 1, wherein the solid pharmaceutical composition is administered to the patient that is diagnosed with hair loss.

17. A method for preparing the solid pharmaceutical composition of claim 1, comprising:
1) mixing the therapeutically effective amount of the minoxidil with a part of the filler in such amount that a volume ratio of the minoxidil to a hydrophobic component of the filler ranges from 1:2 to 1:5 to obtain a material 1;
2) mixing a remainder of the filler with the material 1 in step 1), and then subjecting a resulting mixture to sieving with a granulate machine to obtain a material 2;
3) subjecting the glidant and the pharmaceutically acceptable release controlling agent to sieving with the granulate machine, and then mixing a resulting material with the material 2 in step 2) to obtain a material 3; and
4) adding the lubricant to the material 3 obtained from step 3) and then mixing to obtain the solid pharmaceutical composition.
